(19) United States
(12) Patent Application Publication
(10) Pub. No.: US 2009/0156697 A1
(43) Pub. Date: Jun. 18, 2009

(54) CATALYST COMPOSITIONS AND METHODS FOR ALCOHOL PRODUCTION FROM SYNTHESIS GAS

(75) Inventor: Karl Kharas, Louisville, CO (US)

Correspondence Address: Range Fuels, Inc.;Attn: Ryan O'Connor, 11101 West 120th Ave. Suite 200, Broomfield, CO, 80021, US

(73) Assignee: Range Fuels, Inc., Broomfield, CO (US)

(21) Appl. No.: 12/330,222

(22) Filed: Dec. 8, 2008

Related U.S. Application Data
(60) Provisional application No. 61/013,958, filed on Dec. 14, 2007.
(60) Provisional application No. 61/013,965, filed on Dec. 14, 2007.
(60) Provisional application No. 61/013,975, filed on Dec. 14, 2007.

(51) Int. Cl. B01J 27/232; C01B 17/45; C07C 27/06

(52) U.S. Cl. 518/714; 423/511; 502/174

(57) ABSTRACT

In one aspect of this invention, catalytic compositions produced by calcining intermediates of the formula [NR₄]ₓ[M¹₂M²S₈] are provided, wherein M¹ is Mo or W; M² is Co, Ni, or Pd; x is 2 or 3; and R is a C₃-C₈ alkyl group. Another aspect provides catalytic compositions produced by calcining intermediates of the formula Aₓ[M¹₂M²S₈], wherein A is selected from K, Rb, Cs, Sr, and Ba. Also provided are methods for making the compositions, and methods of using the compositions for the catalytic conversion of syngas into C₁-C₄ alcohols such as ethanol.

### PRIORITY DATA

This patent application claims priority under 35 U.S.C. § 120 from U.S. Provisional Patent Application Nos. 61/013,958; 61/013,965; and 61/013,975, each filed Dec. 14, 2007, and each of which is hereby incorporated herein by reference for all purposes.

### FIELD OF THE INVENTION

The present invention generally relates to catalysts and methods for converting syngas into alcohols, such as ethanol.

### BACKGROUND OF THE INVENTION

Synthesis gas (hereinafter referred to as syngas) is a mixture of hydrogen (H₂) and carbon monoxide (CO). Syngas can be produced, in principle, from virtually any material containing carbon. Carbonaceous materials commonly include fossil resources such as natural gas, petroleum, coal, and lignite, and renewable resources such as lignocellulosic biomass and various carbon-rich waste materials. It is preferable to utilize a renewable resource to produce syngas because of the rising economic, environmental, and social costs associated with fossil resources.

There exist a variety of conversion technologies to turn these feedstocks into syngas. Conversion approaches can utilize a combination of one or more steps comprising gasification, pyrolysis, steam reforming, and/or partial oxidation of a carbon-containing feedstock.

Syngas is a platform intermediate in the chemical and biorefining industries and has a vast number of uses. Syngas can be converted into alkanes, olefins, oxygenates, and alcohols. These chemicals can be blended into, or used directly as, diesel fuel, gasoline, and other liquid fuels. Syngas can also be directly combusted to produce heat and power.

Since the 1920s it has been known that mixtures of methanol and other alcohols can be obtained by reacting syngas over certain catalysts (Forzatti et al., Cat. Rev.—Sci. and Eng. 33(1-2), 109-168, 1991). Fischer and Tropsch observed around the same time that hydrocarbon-synthesis catalysts produced linear alcohols as byproducts (Fischer and Tropsch, Brennst.—Chem. 7:97, 1926).

There is a continuing need for catalyst compositions, and methods for making and using these catalyst compositions, to produce C₁-C₄ alcohols from syngas. An especially preferred alcohol is ethanol, which can replace gasoline and other liquid fuels.

### BRIEF SUMMARY OF THE INVENTION

The present invention provides several aspects that address the aforementioned needs in the art.

In one aspect, the invention provides a compound of the formula [NR₄]ₓ[M¹₂M²S₈], wherein: M¹ is Mo or W; M² is Co, Ni, or Pd; x is 2 or 3; x is 2 when M² is Ni or Pd; x is 3 when M¹ is Mo and M² is Co; and R is a C₃-C₈ alkyl group.

In some embodiments, M¹ is Mo, W, or both Mo and W. In some embodiments, M² is Co, Ni, Pd, two of these, or all of these elements. In some embodiments, R is an n-alkyl group, such as n-butyl, n-pentyl, or n-hexyl.

In some embodiments, the compound has a formula selected from the group consisting of [NR₄]₃[Mo₂CoS₈], [NR₄]₂[Mo₂NiS₈], [NR₄]₃[W₂CoS₈], [NR₄]₂[W₂CoS₈], and [NR₄]₂[W₂NiS₈]. Some compositions include at least two such compounds. For example, some compositions include both [NR₄]₂[Mo₂NiS₈] and [NR₄]₂[Mo₂CoS₈]. Some compositions include both [NR₄]₂[W₂NiS₈] and [NR₄]₂[W₂CoS₈]. Certain compositions include Pd in economically viable amounts. Compositions of the invention can further include a solvent.

In some embodiments, the compound has the formula Aₓ[M¹₂M²S₈], wherein: A is selected from the group consisting of K, Rb, Cs; M¹ is Mo or W; M² is Co, Ni, or Pd; x is 2 or 3; x is 2 when M² is Ni or Pd; and x is 3 when M¹ is Mo and M² is Co. For example, the compound can be selected from the group consisting of A₃[Mo₂CoS₈], A₂[Mo₂NiS₈], A₃[W₂CoS₈], A₂[W₂CoS₈], and A₂[W₂NiS₈].

In other embodiments, the compound has the formula Aₓ[M¹₂M²S₈], wherein: A is selected from the group consisting of Sr and Ba; M¹ is Mo or W; M² is Co, Ni, or Pd; x is 1 or 1.5; x is 1 when M² is Ni or Pd; and x is 1.5 when M¹ is Mo and M² is Co. For example, the compound can be selected from the group consisting of A_{1.5}[Mo₂CoS₈], A[Mo₂NiS₈], A_{1.5}[W₂CoS₈], A[W₂CoS₈], and A[W₂NiS₈].

Certain embodiments employ at least two compounds each in accordance with the foregoing description.

In another aspect, this invention provides a method for generating a catalyst (derived from [R₄N]ₓ[M¹₂M²S₈]) capable of converting syngas into one or more reaction products comprising at least one C₁-C₄ alcohol when in the presence of a catalytic promoter, the method comprising the following steps:

(a) preparing a solution A of a compound of the formula [NH₄]ₓ[M¹S₄] in a first polar solvent, wherein M¹ is Mo or W, and wherein x is 2;

(b) preparing a solution B of a salt or compound of a Group VIII element M² in a second polar solvent, wherein M² is Co, Ni, or Pd, wherein x is 2 when M² is Ni or Pd, and wherein x is 3 when M¹ is Mo and M² is Co;

(c) when x is 3, preparing a solution C containing a reducing agent dissolved in a third polar solvent miscible in the first or second polar solvents;

(d) preparing a solution D of a compound of the formula R₄NZ in a fourth polar solvent, wherein R is a C₃-C₈ alkyl group, and wherein Z is a monovalent anion;

(e) combining solutions A, B, C (if any), and D, to form a precipitate comprising a compound of the formula [R₄N]ₓ[M¹₂M²S₈];

(f) removing the first, second, third (if any), and fourth solvents from the precipitate; and

(g) calcining the precipitate under inert atmosphere to form the catalyst.

In some embodiments, solutions A and B are mixed to form a solution comprising a compound of the formula [NH₄]ₓ[M¹₂M²S₈], prior to mixing with solution C and/or solution D. In some embodiments, solutions B and D are mixed prior to mixing with solution A. Various orders of steps are possible.

Steps (f) and (g) are preferably performed under inert atmospheres. Steps (f) and (g) can be performed without exposing the precipitate to oxygen between the steps. In some embodiments, step (g) is performed in an alcohol-synthesis reactor. Optionally, the catalytic promoter can be combined with the compound [R₄N]ₓ[M¹₂M²S₈] prior to step (g).

In some embodiments, solution D further comprises a base, such as a compound of the formula R₄NZ. Z can be selected from the group consisting of acetate, formate, bicarbonate, and hydroxide. A base can deprotonate ammonium cations and drive precipitation reactions.

The salt of a Group VIII element M² is selected from the group consisting of acetate salt, chloride salt, bromide salt, and nitrate salt.

In other embodiments, the invention provides a method for generating a catalyst, derived from E_{y}[M¹₂M²S₈], the catalyst being capable of converting syngas into one or more reaction products comprising at least one C₁-C₄ alcohol when in the presence of a catalytic promoter, wherein: E is selected from the group consisting of K, Cs, Rb, Sr, and Ba; M¹ is Mo or W; M² is a Group VIII element Co, Ni, or Pd; x is 2 when M² is Ni or Pd; x is 3 when M¹ is Mo and M² is Co; y is x when E is K, Cs, or Rb; and y is x/2 when E is Sr or Ba. This method comprises the following steps:

(a) preparing a solution A of a compound of the formula [NH₄]₂[M¹S₄] in a first polar solvent;

(b) preparing a solution B of a salt or compound of M² in a second polar solvent,

(c) when x is 3, preparing a solution C containing a reducing agent dissolved in a third polar solvent miscible in said first or second polar solvents;

(d) preparing a solution D of a compound of the formula selected from the group consisting of KOH, CsOH, RbOH, Sr(OH)₂, and Ba(OH)₂ in a fourth polar solvent;

(e) combining said solutions A, B, C (if any), and D, to form a precipitate comprising a compound of the formula E_{y}[M¹₂M²S₈];

(f) removing said first, second, third (if any), and fourth solvents from said precipitate; and

(g) calcining said precipitate under inert atmosphere to form said catalyst.

Step (d) can be conducted in the presence of a complexing agent suitable to promote solubilization of the compound provided in step (d). The complexing agent can, for example, be a crown ether.

Steps (f) and (g) are preferably performed under inert atmospheres. In some embodiments, steps (f) and (g) are performed without exposing the precipitate to oxygen between the steps. Step (g) can be performed at a temperature selected from about 350-500° C. for a time selected from about 1-10 hours. In some embodiments, step (g) is performed in an alcohol-synthesis reactor. Optionally, the catalytic promoter can be combined with the compound E_{y}[M¹₂M²S₈] prior to step (g).

Each of the first, second, third, and fourth polar solvents can be separately selected from the group consisting of acetonitrile, dimethylformamide, tetrahydrofuran, C₁-C₄ alcohols, and mixtures thereof. In some embodiments, the first, second, third (if present), and fourth polar solvents are the same. Removal of solvent can be accomplished, for example, by filtration, heating, or some other means.

In some embodiments, the molar ratio of sulfur to the combined total of molybdenum (if present), tungsten (if present), and the Group VIII element(s) in the catalyst is at least about 1.5:1. In certain embodiments, this molar ratio is at least about 1.9:1, 2.0:1, or 2.1:1.

Variations of the invention further include a method of producing at least one C₁-C₄ alcohol from syngas, the method comprising contacting hydrogen and carbon monoxide with a catalyst, produced according to the methods described herein, and combined with a suitable catalytic promoter. Certain embodiments produce ethanol, such as at least 25%, 50%, or more by weight of the total C₁-C₄ alcohols produced.

In some of these variations, the catalyst is exposed to O₂ for less than six hours prior to contacting the catalyst with hydrogen and carbon monoxide. In certain embodiments, the catalyst is exposed to O₂ for less than one hour prior to contacting the catalyst with hydrogen and carbon monoxide. Optionally, the catalyst is not substantially exposed to O₂ prior to contacting the catalyst with hydrogen and carbon monoxide.

Another aspect relates to compositions produced by methods of the invention. In some embodiments, a composition is produced by the method comprising: (a) obtaining a compound having the formula [NR₄]ₓ[M¹₂M²S₈], wherein: (i) M¹ is Mo or W; (ii) M² is Co, Ni, or Pd; (iii) x is 2 or 3; (iv) R is a C₃-C₈ alkyl group; (v) x is 2 when M² is Ni or Pd; and (vi) x is 3 when M¹ is Mo and M² is Co; and (b) calcining the compound under a substantially inert atmosphere.

In some embodiments, the compound obtained or produced in step (a) has a formula selected from the group consisting of [NR₄]₃[Mo₂CoS₈], [NR₄]₂[Mo₂NiS₈], [NR₄]₃[W₂CoS₈], [NR₄]₂[W₂CoS₈], and [NR₄]₂[W₂NiS₈].

Some compositions produced include more than one compound having the formula [NR₄]ₓ[M¹₂M²S₈]. For instance, the at least two compounds can be [NR₄]₂[W₂NiS₈] and [NR₄]₂[W₂CoS₈].

Preferably, solvent in contact with the compound(s) is substantially removed prior to calcining. In some embodiments, the composition is not exposed to oxygen between solvent removal and calcining. Step (b) can be performed, for example, at a temperature selected from about 350-500° C. and a time selected from about 1-10 hours. In some embodiments, step (b) is performed in an alcohol-synthesis reactor. Optionally, a catalytic promoter can be combined, prior to step (b), with the compound [NR₄]ₓ[M¹₂M²S₈] obtained in step (a).

In other embodiments, a composition is produced by the method comprising: (a) obtaining a compound having the formula Aₓ[M¹₂M²S₈], wherein: A is selected from the group consisting of K, Rb, Cs; M¹ is Mo or W; M² is Co, Ni, or Pd; x is 2 or 3; x is 2 when M² is Ni or Pd; x is 3 when M¹ is Mo and M² is Co; and (b) calcining the compound under a substantially inert atmosphere.

In still other embodiments, a composition is produced by the method comprising: (a) obtaining a compound having the formula Aₓ[M¹₂M²S₈], wherein: A is selected from the group consisting of Sr and Ba; M¹ is Mo or W; M² is Co, Ni, or Pd; x is 1 or 1.5; x is 1 when M² is Ni or Pd; and x is 1.5 when M¹ is Mo and M² is Co; and (b) calcining the compound under a substantially inert atmosphere.

Additionally, new and useful compositions can be produced from at least two compounds of formula Aₓ[M¹₂M²S₈], as described above and in more detail herein.

Preferably, solvent in contact with the compound(s) is substantially removed prior to calcining. In some embodiments, the composition is not exposed to oxygen between solvent removal and calcining. Step (b) can be performed, for example, at a temperature selected from about 350-500° C. and a time selected from about 1-10 hours. In some embodiments, step (b) is performed in an alcohol-synthesis reactor. Optionally, a catalytic promoter can be combined, prior to step (b), with the compound Aₓ[M¹₂M²S₈] obtained in step (a).

In some variations, these compositions are capable of catalytically converting syngas into one or more reaction products comprising at least one C₁-C₄ alcohol when in the presence of a catalytic promoter. The catalytic promoter can be K₂CO₃, Cs₂CO₃, or another effective promoter for alcohol synthesis.

In certain embodiments, a composition includes: (a) at least one Group VIB element selected from molybdenum and tungsten; (b) at least one Group VIII element selected from cobalt, nickel, and palladium; and (c) sulfur; wherein the molar ratio of sulfur to the combined total of the at least one Group VIB element and the at least one Group VIII element is at least about 1.9:1; wherein the molar ratio of the at least one Group VIB element to the at least one Group VIII element is 2:1; wherein the composition is essentially free of crystalline phase disulfide of the at least one Group VIII element; and wherein the composition is capable of catalyzing the conversion of syngas into one or more reaction products comprising at least one C₁-C₄ alcohol, such as ethanol, when in the presence of a suitable catalytic promoter.

The present invention will now be described by reference to the following detailed description, which characterizes some preferred embodiments but is by no means limiting.

### DETAILED DESCRIPTION OF SOME EMBODIMENTS OF THE INVENTION

Provided herein are catalytic compositions, intermediates for making the compositions, methods for making the compositions, and methods of using the compositions for the catalytic conversion of syngas into C₁-C₄ alcohols such as ethanol.

“Synthesis gas” and “syngas” are used interchangeably herein, and mean a mixture of H₂ and CO. Syngas may be produced, for example, from fossil resources such as natural gas, petroleum, coal, and lignite, and from renewable resources such as lignocellulosic biomass and various carbon-rich waste materials.

As used herein and in the appended claims, the singular forms “a”, “an” and “the” include plural forms, unless the context clearly dictates otherwise. Unless defined otherwise or clearly indicated by context, all technical and scientific terms used herein have the same meaning as commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Numerical parameters set forth in this specification and the attached claims are approximations that may vary depending at least upon the specific analytical technique. Any numerical value inherently contains certain errors necessarily resulting from the standard deviation found in its respective testing measurements.

As used herein, a catalyst that is “capable of catalytically converting syngas into one or more reaction products comprising at least one C₁-C₄ alcohol when in the presence of a catalytic promoter” refers to a catalyst that produces at least one C₁-C₄ alcohol (e.g., methanol, ethanol, propanol, butanol) when contacted with H₂, CO, and a suitable catalytic promoter under reaction conditions suitable for synthesizing C₁-C₄ alcohols.

In some variations, catalytic intermediate compounds of the formula [NR₄]ₓ[M¹₂M²S₈] are provided, wherein: M¹ is Mo or W; M² is Co, Ni, or Pd; x is 2 or 3; and R is a C₃-C₈ alkyl group. In preferred embodiments, x is 2 when M² is Ni or Pd; and x is 3 when M¹ is Mo and M² is Co. These intermediate compound(s) may be present in one or more solvents, or may be isolated by filtering and/or heating. Calcining the catalytic intermediate compound(s), preferably under inert atmosphere, results in formation of the catalyst that can then be loaded into a reactor. Optionally, the calcination may be performed in an alcohol-synthesis reactor, wherein a catalytic promoter can be mixed with the catalytic intermediate compound(s) prior to calcination.

Mixed intermediate compounds comprising more than one M² (e.g., Co and Ni, Co and Pd, Ni and Pd, or all of Co, Ni, and Pd) may be produced, as discussed below. Mixed intermediate compounds containing both Mo and W may also be produced.

R may be a straight-chain alkyl (n-alkyl) group. R may also be branched (e.g. isopropyl, isobutyl, sec-butyl, etc.) or cyclic (e.g. cyclopropyl, cyclohexyl, cyclopropyl-methyl, etc.), provided that the steric bulk around the nitrogen is not too high to prevent formation of the tetraalkylammonium salt [NR₄]⁺. In some embodiments, R is selected from the group consisting of n-propyl, n-butyl, n-pentyl, n-hexyl, n-heptyl, and n-octyl.

Non-limiting examples of intermediate compounds of the invention include: [NR₄]₃[Mo₂CoS₈], [NR₄]₂[Mo₂NiS₈], [NR₄]₃[W₂CoS₈], [NR₄]₂[W₂CoS₈], and [NR₄]₂[W₂NiS₈], wherein R is a C₃-C₈ alkyl group.

Compositions comprising a single intermediate compound may be produced by methods of the invention. Alternatively, mixed compositions comprising more than one, such as two, three, four or more, of the intermediate compounds may be produced and mixed in any ratio (e.g., when two compositions are mixed, 90:10, 75:25, 50:50, 25:75, or 10:90).

In some embodiments, when Pd and Ni and/or Co are present in the catalytic composition, the Pd is present at a much lower concentration than either Ni or Co. For example, Pd may be present at a ratio of Pd to (Ni+Co) of less than about 50:50, preferably less than about 10:90, and more preferably less than about 1:99. The mixed intermediates may either be created by mixing two or more of the intermediate compounds that are synthesized separately, or may also be synthesized by co-producing two or more intermediate compounds in a single reaction vessel. In the case of co-production of two or more intermediate compounds, in some embodiments, when M² is Co and Ni for the at least two intermediate compounds, then x is 2 and M¹ is W; and when M² is Co and Pd for the at least two intermediate compounds, then M¹ is W.

In another aspect, catalytic intermediate compounds of the formula Aₓ[M¹₂M²S₈] (x=2 or 3) are provided, wherein: M¹ is Mo or W; M² is Co, Ni, or Pd; x is 2 when M² is Ni or Pd; x is 3 when M¹ is Mo and M² is Co; and A is selected from the group consisting of K, Rb, and Cs. In a related aspect, catalytic intermediate compounds of the formula Aₓ[M¹₂M²S₈] (x=1 or 1.5) are provided, wherein: M¹ is Mo or W; M² is Co, Ni, or Pd; x is 1 when M² is Ni or Pd; x is 1.5 when M¹ is Mo and M² is Co; and A is selected from Sr and Ba.

Mixtures comprising both NR₄ and the cation A are also possible. For example, in some embodiments related to sulfided molybdenum/nickel catalysts, KNR₄[Mo₂NiS₈] is produced. Optionally, KNR₄[Mo₂NiS₈] can be co-produced with K₂[Mo₂NiS₈] and [NR₄]₂[Mo₂NiS₈], all of which are effective intermediates suitable for producing effective catalytic compositions.

In some variations of the invention, catalytic compositions are provided, wherein the catalytic composition, when in the presence of a catalytic promoter, is capable of catalyzing the conversion of syngas into one or more reaction products comprising at least one C₁-C₄ alcohol.

In some embodiments, the catalytic composition is produced by the method comprising: (a) obtaining one or more intermediate compounds as described herein; and (b) calcining the compound(s) under inert atmosphere, as described herein.

In some embodiments, the catalytic composition comprises: (a) at least one Group VIB element selected from molybdenum and tungsten; (b) at least one Group VIII element selected from cobalt, nickel, and palladium; and (c) sulfur; wherein the molar ratio of sulfur to the combined total of the at least one Group VIB element and the at least one Group VIII element is preferably at least about 1.9:1. Preferably, the molar ratio of the at least one Group VIB element to the at least one Group VIII element is 2:1. Preferably, the catalytic composition is essentially free of a crystalline disulfide phase of the at least one Group VIII element. In these embodiments, the catalytic composition, when in the presence of a catalytic promoter, is capable of catalyzing the conversion of syngas into reaction products comprising at least one C₁-C₄ alcohol.

The molar ratio of sulfur to the combined total of the at least one Group VIB element and the at least one Group VIII element may be at least about 1.5:1, preferably at least about 1.9:1, more preferably at least about 2.0:1, and most preferably at least about 2.1:1.

The catalytic compositions may comprise one or more metal disulfides, such as molybdenum disulfide or tungsten disulfide. The compositions may comprise one or more metal persulfides, such as one or more of nickel persulfide, cobalt persulfide, and palladium persulfide. As used herein, “persulfide” indicates an anion of the form S₂²⁻.

Non-limiting examples of catalytic compositions of the invention include: 2MoS₂.NiS₂, 2WS₂.NiS₂, 2WS₂.CoS₂, 4WS₂.CoS₂.NiS₂ and 2MoS₂.CoS₂. It is to be understood that a composition with a given empirical formula, e.g. 2MoS₂.NiS₂, may vary in its precise elemental composition, and that the ratios of elements given (2Mo+6S+1Ni) are only approximate.

The catalytic compositions may be essentially free of a crystalline disulfide phase of a Group VIII element. X-ray diffraction is one technique that can be used to make such a determination. Namely, when a composition is essentially free of crystalline disulfide phase of a Group VIII element, the intensity of the most-intense reflection of CoS₂, NiS₂, and PdS₂ will be less than about half that of the most-intense reflection of MoS₂ or WS₂, as measured by X-ray diffraction.

In some embodiments, catalytic compositions include small amounts of a crystalline disulfide phase of a Group VIII element. In these embodiments, the intensity of the most-intense disulfide reflection of CoS₂, NiS₂, and PdS₂, as measured by X-ray diffraction, will be less than about 45%, less than about 35%, or less than about 25% that of the most-intense reflection of MoS₂ or WS₂.

The catalytic compositions of the invention may have a high surface area, such as at least about 10 m²/gram (surface area per gram of total material). In some embodiments, the catalytic compositions have a surface area at least about 25 m²/gram, at least about 50 m²/gram, at least about 75 m²/gram, or at least about 100 m²/gram. Use of increasingly large R groups (in embodiments relating to [NR₄]ₓ[M¹₂M²S₈]) may result in increasing surface area for the resulting catalytic compositions described herein.

The catalytic composition may be produced from a single intermediate compound, or may be produced from more than one intermediate compounds. When making mixed catalytic compositions from more than one intermediate compound, the intermediate compounds may be produced and/or mixed in any ratio prior to calcining, to result in mixed catalytic compositions. In some embodiments, when Pd and Ni and/or Co are present in the catalytic composition, the Pd is present at a much lower concentration than either Ni or Co. The mixed catalytic compositions may either be created by mixing two or more of the catalytic compositions that are synthesized separately, or may be synthesized by co-producing the mixed composition from two or more intermediate compounds in a single reaction vessel. The two or more intermediate compounds may be synthesized separately and then mixed, or they may be co-produced in a single reaction vessel.

When C₁-C₄ alcohols are desired, the catalytic compositions described herein are preferably combined with a suitable catalytic promoter. In the absence of the catalytic promoter, hydrocarbons can be produced in high selectivities. Suitable promoters include alkali promoters such as potassium, cesium, and rubidium, preferably incorporated as anhydrous carbonates, acetates, or hydroxides. Non-limiting examples of suitable promoters include K₂CO₃, K₂C₂H₃, Cs₂CO₃, CsO₂C₂H₃, Rb₂CO₃, RbO₂C₂H₃, and formates or propionates of potassium, rubidium, or cesium.

Without wishing to be bound by theory, one role of a basic promoter is to shift selectivity away from predominantly methane production to provide for selectivity for alcohol synthesis. Another role of the basic promoter may be to suppress acid-catalyzed alcohol dehydration to yield olefins. The promoter may be an inherent constituent of a catalytic intermediate compound, as recited above. Alternatively, or additionally, a promoter may be added to the catalyst, for example, by grinding together with the catalyst. In this case, it is typically convenient to grind, under a substantially inert atmosphere, a salt of a base promoter such as potassium or cesium. It is preferred to grind acetate or carbonate salts with the catalytic materials. Alternatively, the promoter may be mixed with the intermediate compound prior to isolation and calcination of the intermediate to form the catalyst.

The catalyst can take the form of a powder, pellets, granules, beads, extrudates, and so on. When a catalyst support is optionally employed, the support may assume any physical form such as pellets, spheres, monolithic channels, etc. The supports may be coprecipitated with active metal species, or the support may be treated with the catalytic metal species and then used as is or formed into the aforementioned shapes, or the support may be formed into the aforementioned shapes and then treated with the catalytic species.

In embodiments of the invention that employ a catalyst support, the support is preferably (but not necessarily) a carbon-rich material with large mesopore volume, and further is preferably highly attrition-resistant. One carbon support that can be utilized is “Sibunit” activated carbon (Boreskov Inst. of Catalysis, Novosibirsk, Russia) which has high surface area as well as chemical inertness both in acidic and basic media (Simakova et al., Proceedings of SPIE—Volume 5924, 592413, 2005). An example of Sibunit carbon as a catalyst support can be found in U.S. Pat. No. 6,617,464, issued to Manzer.

Methods for making catalytic compositions described herein are provided in some variations. These catalytic compositions, when in the presence of a catalytic promoter, are capable of catalyzing the conversion of syngas into one or more reaction products comprising at least one C₁-C₄ alcohol.

In some embodiments, a catalytic composition is produced by the method comprising: (a) obtaining one or more intermediate compounds as described herein, and (b) calcining the compound(s) under inert atmosphere, as described herein.

In some embodiments, the method for making a catalytic composition derived from a precipitate of the formula [R₄N]ₓ[M¹₂M²S₈] (x=2 or 3) comprises the following steps:

(a) preparing a solution A of a compound of the formula [NH₄]₂[M¹S₄] in a first polar solvent, wherein M¹ is Mo or W;

(b) preparing a solution B of a salt of a Group VIII element M² in a second polar solvent, wherein M² is Co, Ni, or Pd, wherein x is 2 when M² is Ni or Pd, and wherein x is 3 when M¹ is Mo and M² is Co;

(c) when x is 3, preparing a solution C containing a reducing agent dissolved in a third polar solvent miscible in the first or second polar solvents;

(d) preparing a solution D of a compound of the formula R₄NZ in a fourth polar solvent, wherein R is a C₃-C₈ alkyl group, and wherein Z is a monovalent anion;

(e) combining solutions A, B, C (if present), and D, to form a precipitate comprising a compound of the formula [R₄N]ₓ[M¹₂M²S₈];

(f) removing the first, second, third (if present), and fourth solvents from the precipitate; and

(g) calcining the precipitate under inert atmosphere to form the catalyst. Steps (f) and (g) are preferably (but not necessarily) performed without exposing the precipitate to oxygen between the steps.

The solution C, used when x is 3, can contain a reducing agent such as a soluble salt of the [SC₆H₅]⁻ anion, a salt of the BH₄⁻ anion, or hydrazine. Step (c) is not necessary when x is 2. In some embodiments, in step (e), a non-polar solvent is added to induce or aid precipitation. This non-polar solvent is preferably deoxygenated and free of water, and it is preferably miscible in at least one, more preferably at least two, and most preferably all of the first, second, third (if present) and fourth polar solvents.

In some embodiments, solutions A and B are mixed to form a solution comprising a compound of the formula [NH₄]ₓ[M¹₂M²S₈], prior to mixing with solution C and/or solution D. In some embodiments, solution B and solutions C and/or D are mixed prior to mixing with solution A. These particular embodiments may be useful, for example, when the ammonium salts [NH₄]ₓ[M¹₂M²S₈] are not soluble.

Optionally, a base may be added to solution D. For example, when smaller alkyl chains (e.g. propyl) are used as a precipitant (e.g. for [MoS₄]²⁻), tetraalkylammonium hydroxide may be used. Using such an approach, ammonium is deprotonated and cannot compete for the anion, so precipitation is driven forward. The base may be added to solution D, or the tetraalkylammonium hydroxide may be used to prepare solution D.

Monovalent anion Z may be, for example, acetate, formate, hydroxide, or bicarbonate. The salt of a Group VIII element M² may be, for example, an acetate, chloride, bromide, or nitrate salt. In some embodiments, the salt of a Group VIII element M² is an acetate, chloride, or bromide salt.

Examples of suitable polar solvents for the first, second, third, and fourth polar solvents include, for example, acetonitrile, dimethylformamide, tetrahydrofuran, C₁-C₄ alcohols, water, and mixtures thereof. The first, second, third, and fourth polar solvents may be the same, or they may be different. In some embodiments, the first, second, third, or fourth polar solvent is methanol, acetonitrile, or a mixture of methanol and acetonitrile. Water is a less-preferred solvent.

A base, such as KOH, may be added to solutions containing [NH₄]ₓ[M¹₂M¹₁S₈] (x=2 or 3) yielding solutions containing Kₓ[M¹₂M²₁S₈] together with H₂O and NH₃. If it is desired to separate the Kₓ[M¹₂M²S₈] compound(s) from solution by filtering, a non-polar solvent miscible in solvent mixtures A, B, and C (if present) may be added to induce precipitation of the Kₓ[M¹₂M²₁S₈]. In analogous fashion, other salts of [M¹₂M²S₈] may be made using hydroxides of rubidium, cesium, barium, and so forth. The mixed solvent (solutions A, B, and C, if present) and additional non-polar solvent (such as hexane, cyclohexane, decane, toluene, ethylbenzene, and xylenes) may, after separation from the salt of [M¹₂M²₁S₈], be separated by, for instance, distillation and reused. These compositions effectively incorporate catalyst promoters during synthesis.

The first, second, third (if present), and fourth solvents may be removed, for example, by filtering and/or heating. In some embodiments, the filtering is performed under air. In some embodiments, the filtering is performed under inert atmosphere. For example, for compounds comprising Ni, filtering under air or under inert atmosphere may be used. For compounds comprising Mo and Co, and for compounds comprising [W₂CoS₈]³⁻, filtering under inert atmosphere is preferred. Generally, the filter cake is not truly dried but remains moist with mother liquor from the initial suspension. Final drying of the catalyst, by either filtering or heating, may be performed under air or under inert atmosphere. In general, the temperature used to remove the solvent and dry the compound can be a temperature near or slightly above the atmospheric-pressure boiling point of the solvent. Between drying the precipitate and calcining, the precipitate may be exposed to air, or may be kept under inert atmosphere. In some embodiments, the precipitate is exposed to air between drying and calcination. In preferred embodiments, the precipitate is kept under inert atmosphere between drying and calcination.

The catalytic compositions are produced by calcining one or more of the intermediate compounds. The calcination temperature and time may be selected so as to maximize production of persulfide, and minimize thermal reduction and loss of sulfur. For example, preferred calcination of [NR₄]₂[Mo₂NiS₈] will result in a catalyst having an empirical formula of about 2MoS₂.NiS₂. If calcination, however, runs for too long or too high of a temperature, more sulfur will be lost, resulting in a catalyst with an empirical formula closer to 2MoS₂.NiS. Further, drying the compounds under air, followed by calcination under inert atmosphere, can also be associated with the loss of more sulfur, resulting in compositions with an empirical formula closer to MoS₂.NiS, whereas drying and calcination under inert atmosphere will yield 2MoS₂.NiS₂. In another example, calcination of a mixture of [NR₄]₂[W₂CoS₈] and [NR₄]₂[W₂NiS₈] can be conducted to yield a composition with an empirical formulation of about 4WS₂.CoS₂.NiS₂; calcination for too long of a time or too high of a temperature can result in larger amounts of thermal reduction and a composition approximating 4WS₂.CoS.NiS. These latter compositions (2MoS₂.NiS and 4WS₂.CoS.NiS) will be catalytically active for alcohol production, but will typically have reduced catalytic activity. The calcination may be performed at a temperature of, for example, about 350-500° C. for about, for example, 1-10 hours.

In some embodiments, the method for making a catalytic composition comprises similar steps as recited herein above, to produce a precipitate comprising compound of the M¹₂M²S₈ anion with an effective cation. These catalytic intermediate compounds can generally have the formula Aₓ[M¹₂M²S₈] (x=2 or 3) when M¹ is Mo or W; M² is Co, Ni, or Pd; x is 2 when M² is Ni or Pd; x is 3 when M¹ is Mo and M² is Co; and A is selected from the group consisting of K, Rb, and Cs. Or, these catalytic intermediate compounds can have the formula Aₓ[M¹₂M²S₈] (x=1 or 1.5) when M¹ is Mo or W; M² is Co, Ni, or Pd; x is 1 when M² is Ni or Pd; x is 1.5 when M¹ is Mo and M² is Co; and A is selected from Sr and Ba.

The catalytic compositions described herein, in certain variations of the invention, may be used to produce C₁-C₄ alcohols (methanol, ethanol, propanol, and butanol, including all isomers) from syngas by contacting hydrogen, carbon monoxide, and a catalytic promoter with the catalyst. Methods of synthesizing alcohols from syngas using catalysts and catalyst promoters are described in, for example, U.S. patent application Ser. No. 12/166,203, filed Jul. 1, 2008 and which is hereby incorporated by reference herein in its entirety for all purposes.

In general, a reactor (such as a fixed-bed reactor with continuous gas flow) may be loaded with a catalytic composition (described herein) and a catalytic promoter (such as Cs₂CO₃ or K₂CO₃). The catalyst may be loaded into the reactor with minimum exposure to air. In some embodiments, the catalyst is exposed to O₂ for less than about six hours, less than about an hour, less than about 10 minutes, or less than about 1 minute prior to contacting the catalyst with hydrogen and carbon monoxide. In some embodiments, the catalyst is not exposed to O₂ prior to contacting the catalyst with hydrogen and carbon monoxide. In some embodiments, the catalyst is produced from the intermediate directly in the reactor. After loading the catalytic composition and the catalytic promoter, the reactor is charged with syngas (pressurized, at e.g. 1500 psi), and the reactor is taken to operating conditions appropriate for the synthesis of alcohols.

In some embodiments, conditions effective for producing alcohols from syngas include a feed hydrogen/carbon monoxide molar ratio (H₂/CO) from about 0.2-4.0, preferably about 0.5-2.0. These ratios are indicative of certain embodiments and are not limiting. It is possible to operate at feed H₂/CO ratios less than 0.2 as well as greater than 4, including 5, 10, or even higher. It is well-known that high H₂/CO ratios can be obtained with extensive steam reforming and/or water-gas shift in operations prior to the syngas-to-alcohol reactor.

In some embodiments, conditions effective for producing alcohols from syngas include reactor temperatures from about 200-400° C., preferably about 250-350° C.; and reactor pressures from about 20-500 atm, preferably about 50-200 atm or higher. Generally, productivity increases with increasing reactor pressure. Temperatures and pressures outside of these ranges can be employed.

In some embodiments, conditions effective for producing alcohols from syngas include average reactor residence times from about 0.1-10 seconds, preferably about 0.5-2 seconds. “Average reactor residence time” is the mean of the residence-time distribution of the mobile-phase reactor contents under actual operating conditions. Catalyst space times and/or catalyst contact times can also be calculated by a skilled artisan and these times will typically also be in the range of 0.1-10 seconds, although it will be appreciated that it is certainly possible to operate at shorter or longer times.

In general, the specific selection of catalyst configuration (geometry), H₂/CO ratio, temperature, pressure, residence time (or feed rate), and other reactor-engineering parameters will be selected to provide an economical process. These parameters are not regarded as critical to the present invention. It is within the ordinary skill in the art to experiment with different reactor conditions to optimize selectivity to a particular product or some other parameter.

Product selectivities can be calculated on a carbon-atom basis. “Carbon-atom selectivity” means the ratio of the moles of a specific product to the total moles of all products, scaled by the number of carbon atoms in the species. This definition accounts for the mole-number change due to reaction. The selectivity Sⱼ to general product species Cₓ_{ⱼ}H_{y}_{ⱼ}O_{z}_{ⱼ} s

$${S}_{j} = \frac{{x}_{j} {F}_{j}}{{\sum }_{i} {x}_{i} {F}_{i}}$$

wherein Fⱼ is the molar flow rate of species j which contains xⱼ carbon atoms. The summation is over all carbon-containing species Cₓ_{ᵢ}H_{y}_{ᵢ}O_{z}_{ᵢ} produced in the reaction.

In some embodiments, wherein all products are identified and measured, the individual product selectivities sum to unity (plus or minus analytical error). In other embodiments, wherein one or more products are not identified in the exit stream, the selectivities can be calculated based on what products are in fact identified, or instead based on the conversion of reactants. In the latter case, the selectivities may not sum to unity if there is some mass imbalance. This method can, however, be preferable as it tends to determine more accurate selectivities to identified products when it is suspected that at least one reaction product is not measured.

“CO₂-free carbon-atom selectivity” or “CO₂-free selectivity” mean the percent of carbon in a specific product with respect to the total carbon converted from carbon monoxide to some product other than carbon dioxide. It is the same equation above for Sⱼ, except that i≠CO₂ and j≠CO₂.

In various embodiments of the present invention, the product stream from the reactor may be characterized by CO₂-free selectivities of about 10-40% to methanol and about 20-60% or higher to ethanol. In some preferred embodiments, the ethanol CO₂-free selectivity is higher, preferably substantially higher, than the methanol CO₂-free selectivity, such as a CO₂-free selectivity ratio of ethanol/methanol in the product of about 1.0, 1.5, 2.0, 2.5, 3.0, or higher. The product stream can also contain more methanol than ethanol, on either a mole basis or a carbon-atom basis, in certain embodiments. The CO₂-free selectivity ratio of ethanol to all other alcohols is preferably at least 1, more preferably at least 2.

The method produces one or more reaction products, including at least one C₁-C₄ alcohol. In some embodiments, at least about 25% of the total C₁-C₄ alcohols produced is ethanol. In certain embodiments, at least about 50% of the total C₁-C₄ alcohols produced is ethanol.

The product stream from the reactor may include up to about 25% CO₂-free selectivity to C₃, alcohols, and up to about 10% to other non-alcohol oxygenates such as aldehydes, esters, carboxylic acids, and ketones. These other oxygenates can include, for example, acetone, 2-butanone, methyl acetate, ethyl acetate, methyl formate, ethyl formate, acetic acid, propanoic acid, and butyric acid.

This invention has been described and specific examples of the invention have been portrayed. While the invention has been described in terms of particular variations, those of ordinary skill in the art will recognize that the invention is not limited to the variations described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above.

All publications, patents, and patent applications cited in this specification are incorporated herein by reference in their entirety as if each publication, patent, or patent application were specifically and individually put forth herein.

In this detailed description, reference has been made to multiple embodiments. Other embodiments that do not provide all of the features and advantages set forth herein may be utilized, without departing from the spirit and scope of the present invention. To the extent there are variations of the invention, which are within the spirit of the disclosure or equivalent to the inventions found in the claims, it is intended that this patent will cover those variations as well.

What is claimed is:

1. A method for generating a catalyst derived from [R₄N]ₓ[M¹₂M²S₈], said catalyst capable of converting syngas into one or more reaction products comprising at least one C₁-C₄ alcohol when in the presence of a catalytic promoter, said method comprising:
(a) preparing a solution A of a compound of the formula [NH₄]₂[M¹S₄] in a first polar solvent, wherein M¹ is Mo or W;
(b) preparing a solution B of a salt or compound of a Group VIII element M² in a second polar solvent, wherein M² is Co, Ni, or Pd, wherein x is 2 when M² is Ni or Pd, and wherein x is 3 when M¹ is Mo and M² is Co;
(c) when x is 3, preparing a solution C containing a reducing agent dissolved in a third polar solvent miscible in the first or second polar solvents;
(d) preparing a solution D of a compound of the formula R₄NZ in a fourth polar solvent, wherein R is a C₃-C₈ alkyl group, and wherein Z is a monovalent anion;
(e) combining solutions A, B, C (if any), and D, to form a precipitate comprising a compound of the formula [R₄N]ₓ[M¹₂M²S₈];
(f) removing the first, second, third (if any), and fourth solvents from the precipitate; and
(g) calcining the precipitate under inert atmosphere to form the catalyst.

2. The method of claim 1, wherein solution A and solution B are mixed to form a solution comprising a compound of the formula [NH₄]ₓ[M¹₂M²S₈], prior to mixing with solution C and/or solution D.

3. The method of claim 1, wherein solution B and solution D are mixed prior to mixing with solution A.

4. The method of claim 1, wherein step (f) is performed under inert atmosphere.

5. The method of claim 1, wherein step (g) is performed under inert atmosphere.

6. The method of claim 1, wherein steps (f) and (g) are performed without exposing said precipitate to oxygen between said steps.

7. The method of claim 6, wherein step (g) is performed in an alcohol-synthesis reactor.

8. The method of claim 6, wherein said catalytic promoter is combined with said compound [R₄N]ₓ[M¹₂M²S₈] prior to step (g).

9. The method of claim 1, wherein solution D further comprises a base.

10. The method of claim 1, wherein Z is selected from the group consisting of hydroxide, acetate, formate, and bicarbonate.

11. The method of claim 1, wherein said salt of a Group VIII element M² is selected from the group consisting of acetate salt, chloride salt, bromide salt, and nitrate salt.

12. A method for generating a catalyst derived from E_{y}[M¹₂M²S₈], said catalyst capable of converting syngas into one or more reaction products comprising at least one C₁-C₄ alcohol when in the presence of a catalytic promoter, wherein:
E is selected from the group consisting of K, Cs, Rb, Sr, and Ba;
M¹ is Mo or W;
M² is a Group VIII element Co, Ni, or Pd;
x is 2 when M² is Ni or Pd;
x is 3 when M¹ is Mo and M² is Co;
y is x when E is K, Cs, or Rb; and
y is x/2 when E is Sr or Ba;
said method comprising:
(a) preparing a solution A of a compound of the formula [NH₄]₂[M¹S₄] in a first polar solvent;
(b) preparing a solution B of a salt or compound of M² in a second polar solvent,
(c) when x is 3, preparing a solution C containing a reducing agent dissolved in a third polar solvent miscible in said first or second polar solvents;
(d) preparing a solution D of a compound of the formula selected from the group consisting of KOH, CsOH, RbOH, Sr(OH)₂, and Ba(OH)₂ in a fourth polar solvent;
(e) combining said solutions A, B, C (if any), and D, to form a precipitate comprising a compound of the formula E_{y}[M¹₂M²S₈];
(f) removing said first, second, third (if any), and fourth solvents from said precipitate; and
(g) calcining said precipitate under inert atmosphere to form said catalyst.

13. The method of claim 12, wherein said step (d) is conducted in the presence of a complexing agent suitable to promote solubilization of said compound provided in step (d).

14. The method of claim 13, wherein said complexing agent is a crown ether.

15. The method of claim 12, wherein step (f) is performed under inert atmosphere.

16. The method of claim 12, wherein step (g) is performed under inert atmosphere.

17. The method of claim 12, wherein steps (f) and (g) are performed without exposing said precipitate to oxygen between said steps.

18. The method of claim 17, wherein step (g) is performed in an alcohol-synthesis reactor.

19. The method of claim 17, wherein said catalytic promoter is combined with said compound E_{y}[M¹₂M²S₈] prior to step (g).

20. The method of either of claims 1 or 12, wherein said first polar solvent is selected from the group consisting of acetonitrile, dimethylformamide, tetrahydrofuran, C₁-C₄ alcohols, and mixtures thereof.

21. The method of either of claims 1 or 12, wherein said second polar solvent is selected from the group consisting of acetonitrile, dimethylformamide, tetrahydrofuran, C₁-C₄ alcohols, and mixtures thereof.

22. The method of either of claims 1 or 12, wherein said third polar solvent, when present, is selected from the group consisting of acetonitrile, dimethylformamide, tetrahydrofuran, C₁-C₄ alcohols, and mixtures thereof.

23. The method of either of claims 1 or 12, wherein said fourth polar solvent is selected from the group consisting of acetonitrile, dimethylformamide, tetrahydrofuran, C₁-C₄ alcohols, and mixtures thereof.

24. The method of either of claims 1 or 12, wherein said first, second, third (if present), and fourth polar solvents are the same.

25. The method of either of claims 1 or 12, comprising removal of solvent by filtration.

26. The method of either of claims 1 or 12, comprising removal of solvent by heating.

27. The method of either of claims 1 or 12, wherein step (g) is performed at a temperature selected from about 350-500° C. for a time selected from about 1-10 hours.

28. The method of either of claims 1 or 12, wherein the molar ratio of sulfur to the combined total of molybdenum, if present, tungsten, if present, and said Group VIII element(s) in the catalyst is at least about 1.5:1.

29. The method of claim 28, wherein said molar ratio is at least about 1.9:1.

30. The method of claim 29, wherein said molar ratio is at least about 2.0:1.

31. The method of claim 30, wherein said molar ratio is at least about 2.1:1.

32. A method of producing at least one C₁-C₄ alcohol from syngas, said method comprising contacting hydrogen and carbon monoxide with a catalyst produced according to the method of either of claims 1 or 12 and combined with a suitable catalytic promoter.

33. The method of claim 32, wherein said catalyst is exposed to O₂ for less than six hours prior to contacting said catalyst with hydrogen and carbon monoxide.

34. The method of claim 32, wherein said catalyst is exposed to O₂ for less than one hour prior to contacting said catalyst with hydrogen and carbon monoxide.

35. The method of claim 32, wherein said catalyst is not substantially exposed to O₂ prior to contacting said catalyst with hydrogen and carbon monoxide.

36. The method of claim 32, wherein at least 25% of the total C₁-C₄ alcohols produced is ethanol.

37. The method of claim 36, wherein at least 50% of the total C₁-C₄ alcohols produced is ethanol.
